(19) United States
(12) Patent Application Publication
(10) Pub. No.: US 2026/0218105 A1
(43) Pub. Date: Jul. 30, 2026

(54) An Apparatus and Method for Culturing Cells

(71) Applicant: CAMBRIDGE ENTERPRISE LIMITED, Cambridge Cambridgeshire (GB)

(72) Inventors: Gengyao WEI, Cambridge Cambridgeshire (GB); Fraser Anton BIRKS, Cambridge Cambridgeshire (GB); Daniel Victor BAX, Cambridge Cambridgeshire (GB); Serena BEST, Cambridge Cambridgeshire (GB); Ruth Elizabeth CAMERON, Cambridge Cambridgeshire (GB); Malavika NAIR, Cambridge Cambridgeshire (GB)

(21) Appl. No.: 19/147,259

(22) PCT Filed: Jan. 12, 2024
(86) PCT No.: PCT/GB2024/050083

§ 371 Date: Jul. 10, 2025

(30) Foreign Application Priority Data
Jan. 13, 2023 (GB) 2300531.7

(51) Int. Cl. C12M 1/12; B33Y 80/00; C12M 1/00; C12M 1/42

(52) U.S. Cl. CPC C12M 25/02; C12M 23/20; C12M 35/04; B33Y 80/00

(57) ABSTRACT

The present disclosure provides a cell culturing apparatus (1) comprising an auxetic mesh (2) and a substrate (6) for culturing cells. The substrate (6) is attached to the auxetic mesh (2) such that a change in at least biaxial strain in the substrate (6) and/or the auxetic mesh (2) occurs by the application of a uniaxial force to the auxetic mesh (2). The present disclosure also provides a method of for culturing cells and a method of manufacturing a cell culturing apparatus.

The invention relates generally to an apparatus and method for culturing cells. More particularly, the invention relates to a cell culturing apparatus, a method for culturing cells and a method of manufacturing the cell culturing apparatus.

### BACKGROUND

Mechanobiology and related fields have demonstrated that cells can sense and respond to the mechanical properties of their physical environment such as an imposed mechanical force. For example, in mesenchymal stem cells (MSCs) differentiation can be guided by the stiffness of the substrate in which the cells are cultured. Different mechanical forces including uniaxial tension, biaxial tension and shear forces can be imposed on cells of different phenotypes. These forces are intended to mimic a certain mechanical environment that different cells would experience in the body. Applying a force to a substrate in which a cell is cultured is also applicable to the fields of biomaterials and tissue engineering. For example, a tissue culture substrate design may be focused on mimicking the natural in vivo environment. In general, the closer the substrate design is to the natural environment, the more positively the cells would be expected to respond. Closely mimicking the natural environment would include applying physiologically-relevant forces to substrates for culturing cells.

Known techniques of applying forces to a substrate include creating uniaxial strain in cell-seeded substrates. These are techniques are often expensive and limited to particular types of substrates. Additionally, uniaxial strain in a substrate does not closely mimic the natural environment of many cells.

A further known technique is for creating a biaxial strain in a substrate. This technique utilises a flexible silicone membrane (e.g. polydimethylsiloxane, PDMS) coated with cell-anchoring proteins. This membrane provides a thin film which is deformed to strain the cultured cells. However, such systems can often be expensive and can be difficult to control the strain of the membrane. The technique is not suitable for applying strain to a three-dimensional (3D) cell substrate with a complex cell spatial distribution it can only accommodate straining of single layer of cells. The ability to strain 3D cell substrates is useful for tissue engineering, as a 3D cell spatial distribution more closely reflects the in vivo cell condition.

It is an object of embodiments of the invention to at least mitigate one or more problems associated with known arrangements. In particular, the present invention provides a low cost and versatile cell culturing apparatus which can mimic natural environments that different cells would experience in the body.

### SUMMARY OF THE DISCLOSURE

According to an aspect of the invention, there is provided a cell culturing apparatus. The cell culturing apparatus comprises an auxetic mesh; and a substrate for culturing cells, the substrate being attached to the auxetic mesh such that a change in at least biaxial strain in the substrate occurs by the application of a uniaxial force to the auxetic mesh. In particular, the substrate is attached to the auxetic mesh such that a change in at least a biaxial strain in the substrate and/or the auxetic mesh occurs by a corresponding change in at least biaxial strain in the auxetic mesh due to the application of a uniaxial force to the auxetic mesh.

The invention is based on the surprising finding that two-dimensional (2D) and three-dimensional (3D) auxetic structures such as meshes can be used to translate a uniaxial force into at least a biaxial strain which can be applied cells cultured on a cell substrate. The production of such devices is relatively easy and extremely cheap. A force can be applied to such devices with simple mechanical setups to achieve static and cyclic loading. Potential applications of such devices can range from academic laboratory equipment for advanced tissue engineering studies to next generation pharmaceutical drug testing platforms. Such devices have the potential to replicate many physiologically relevant straining situations to biological cells, tissues and organoids. For example, the auxetic mesh and substrate can be selected to replicate different physiologically environments.

The cell culturing apparatus may be an ex vivo or in vitro cell culturing apparatus.

In certain embodiments, the substrate may comprise a three-dimensional cell culture substrate.

In certain embodiments, the auxetic mesh may comprise a re-entrant honeycomb structure. In certain embodiments, the auxetic mesh may comprise a Poisson's ratio of −0.7 to −1.0.

In certain embodiments, the auxetic mesh may comprise a repetition of identical unit cells. In certain embodiments, the auxetic mesh may comprise a repetition of at least two different types of unit cells.

In certain embodiments, the auxetic mesh may be embedded in the substrate. Embedding the auxetic mesh in the substrate may ensure that the biaxial strain field is distributed evenly to the substrate to ensure a predictable and uniform resultant strain field.

In certain embodiments, the auxetic mesh may extend beyond at least two opposing surfaces of the substrate. In certain embodiments, the auxetic mesh may comprise poly-lactic acid.

In certain embodiments, the substrate may comprise a porous structure.

The substrate may have a positive Poison's ratio.

In certain embodiments, the substrate may comprise or essentially consist of a material which is different to the material that the auxetic mesh comprises or essentially consists of.

In certain embodiments, the substrate may comprise a protein and/or a polysaccharide. Suitably, the protein may be an extracellular matrix protein. Suitably the extracellular matrix protein may be selected from the group consisting of collagen elastin, laminin, and fibronectin.

In certain embodiments, the substrate may comprise MatriGel™.

In certain embodiments, the substrate may be crosslinked.

The substrate may be configured to mimic a physiological environment. The physiological environment is an organ specific environment. The organ specific environment is a lung environment.

In certain embodiments, the substrate may comprise cells seeded therein and/or thereon. The cells may comprise a tissue and/or organoid.

The auxetic mesh may not comprise cells seeded therein or thereon.

The substrate may comprise or essentially consist of a single piece of porous substrate material. For example, the substrate may consist of a collagen tissue engineering scaffold. The auxetic mesh may bisect the single piece of porous substrate material.

The substrate may be defined by a shape which does not conform to the auxetic mesh. For example, the substrate may be cuboidal or be cylindrical in shape while the auxetic mesh may have a re-entrant honeycomb structure.

The auxetic mesh may have a thickness which is less than the thickness of the substrate. The auxetic mesh thickness may be less than 90%, 80%, 70% or 60% of the thickness of the substrate. In some examples, the auxetic mesh thickness may be less than 50%, 40%, 30%, 20% or 10% of the thickness of the substrate. For example, the auxetic mesh thickness may be between 0.2 mm and 0.8 mm while the substrate thickness may be 2 mm to 8 mm.

In any of the above-described embodiments, the auxetic mesh may comprises a two-dimensional auxetic mesh or a three-dimensional auxetic mesh.

According to another aspect of the invention, there is provided a method for culturing cells. The method comprising: providing the above-described cell culturing apparatus; and applying a force to the auxetic mesh. Suitably, the substrate may comprise cells seeded therein and/or thereon.

In certain embodiments, the method for culturing cells may comprise: providing the above-described cell culturing apparatus, applying cells to the substrate, and applying a force to the auxetic mesh.

In certain embodiments, the method may further comprise: applying cells to the substrate; disposing the substrate in a culture medium; and maintaining the substrate under conditions suitable for culturing the cells.

In the method, the force applied to the auxetic mesh may comprise a physiologically-mimetic force. In the method, the force may comprise a uniaxial force.

According to another aspect of the invention, there is provided a method of manufacturing a cell culturing apparatus. The method comprising: providing an auxetic mesh; and attaching a substrate for culturing cells to the auxetic mesh such that a change in a biaxial strain in the substrate and/or the auxetic mesh occurs by the application of a uniaxial force to the auxetic mesh.

In the method, providing an auxetic mesh may comprise 3D-printing the auxetic mesh.

In the method, attaching the substrate to the auxetic mesh may comprise suspending a portion of the auxetic mesh with a mould and forming the substrate around the suspended portion of the auxetic mesh.

Herein is also provided, an auxetic mesh for cell culture comprising: a re-entrant honeycomb structure and a Poisson's ratio of −0.7 to −1.0. The mesh may be formed from poly-lactic acid.

### BRIEF DESCRIPTION OF THE DRAWINGS

Embodiments of the invention will now be described, by way of example only, with reference to the accompanying figures, in which:

FIG. 1 is a cell culturing apparatus according an embodiment of the invention;

FIG. 2a is a mesh of the cell culturing apparatus of FIG. 1 and FIG. 2b is a unit cell of the mesh;

FIG. 3 is an assembly for using the cell culturing apparatus of FIG. 1 according an embodiment of the invention;

FIG. 4 is an apparatus for manufacturing the cell culturing apparatus;

FIG. 5 is the apparatus of FIG. 4 during manufacture of the cell culturing apparatus;

FIG. 6 is wet condition tensile testing setup for a substrate;

FIG. 7 shows data for how the final freezing temperature and air insulation affects the porous architecture of the collagen scaffolds. When freeze dried with air insulation, increase in final freezing temperature resulted in higher pore size shown in FIG. 7a, unchanged degree of anisotropy shown in FIG. 7b, and lower percolation diameters shown in FIG. 7c. The data shown is averaged from VOIs above and below the mesh; all freeze dried with a mould height of 6 mm;

FIG. 8 shows data for how the porous architecture of the collagen scaffolds was is influenced by mould height and air insulation. Freeze drying with air insulation led to higher pore size shown in FIG. 8a, higher degree of anisotropy shown in FIG. 8b, and lower percolation diameters shown in FIG. 8c. The data averaged from VOIs above and below the mesh; all freeze dried at a final freezing temperature of −10° C.;

FIG. 9 shows data indicating how the incorporation of the mesh in the scaffolds affects the porous architecture as the pore size and degree of anisotropy above and below the mesh. FIGS. 9a and 9b show data for the pore size and degree of anisotropy against final freezing temperature of scaffolds made with 6 mm mould height with air insulation. FIGS. 9c and 9d show pore size and degree of anisotropy varied against mould height of scaffolds made with a −10° C. final freezing temperature with air insulation;

FIG. 10 shows a stress-strain curve for a collagen scaffold and shows data for how mechanical properties of the collagen scaffold vary with pore size. In particular, FIG. 10 shows that the tensile moduli decreases with increasing pore size and that ultimate tensile strain increases with increasing pore size;

FIG. 11 shows a stress-strain curve for an auxetic mesh and shows data for how the tensile modulus of the mesh decreases linearly with the vertical strut length. The ultimate tensile stress remains similar across the meshes;

FIG. 12 shows data for the measured Poisson's ratio of meshes of increasing strut lengths and the theoretical Poisson's ratio for the meshes calculated based on an analytical solution; and

FIG. 13 shows data demonstrating how straining of the mesh induces biaxial strains on the collagen scaffold substrate on a cell culture apparatus.

FIG. 14 shows the theoretical Poisson's ratio contour of −1 plotted against h/l, tₕ/h, and tₗ/l projected with a projected colour map showing the measured Poisson's ratio values of simulated constrained auxetic meshes. A −1 Poisson's ratio was only observed for constrained mesh at the low tₕ/h and tₗ/l corner.

FIG. 15 shows spatial distribution of pore deformation at different heights above the auxetic mesh when a linear strain of 0.03 was applied to the auxetic mesh. (a) (b) 0.5-1 mm; (c) (d) 1-1.5 mm; (e) (f) 1.5-2 mm; (g) (h) 2-2.5 mm. (a) (c) (e) (g) demonstrated colour maps showing percentage volume change across the scaffold. (b) (d) (f) (h) demonstrated the line profile of volume change across the lines indicated with an arrow at X=0.094 m as indicated.

FIG. 16a shows data demonstrating the aspect ratio of human dermal fibroblasts exposed to 5% strain at five timepoints against the aspect ratio of an non-strained control group. (*): p<0.05, (**): p<0.01, (***): p<0.001, n=3.

FIG. 16b shows data demonstrating the averaged alignment angle of the embedded cells at five timepoints during straining. n=3.

### DETAILED DESCRIPTION

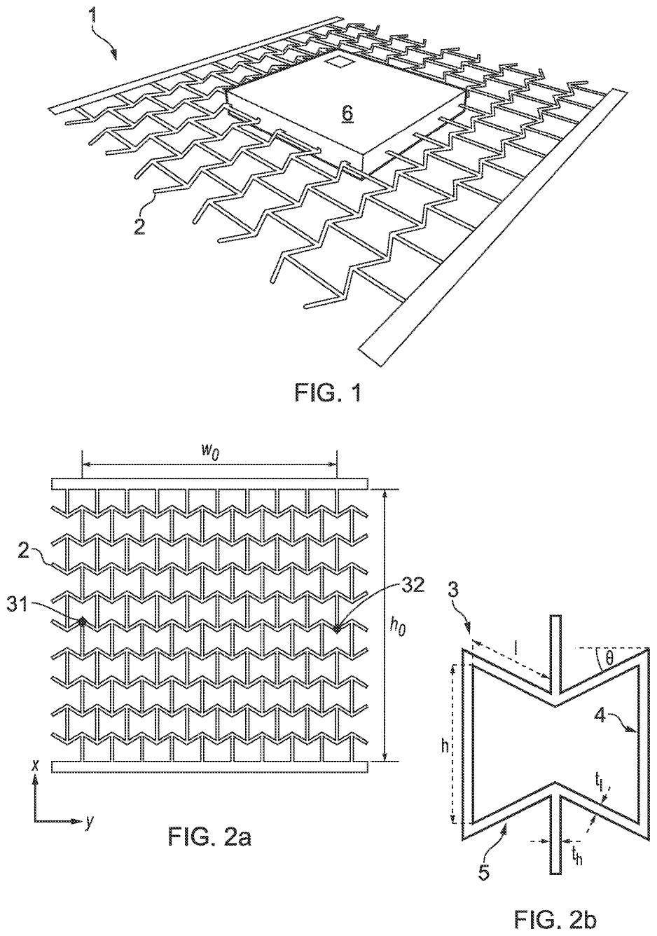

FIG. 1 shows a cell culturing apparatus 1 according to an embodiment of the invention. The apparatus 1 comprises an auxetic mesh 2. In the embodiment shown in FIG. 1, the auxetic mesh 2 comprises a two-dimensional (2D) mesh and it is illustrated as being in the x-y plane.

An auxetic mesh is a mesh which has a negative Poisson's ratio. Therefore, in the embodiment shown in FIG. 1, when a tension force is applied to stretch the auxetic mesh 2 in a direction parallel to the x axis, the mesh 2 also stretches in a direction perpendicular to the force i.e. along the y-axis shown in FIG. 1. Thus, a uniaxial tension force applied to the auxetic mesh 2 causes biaxial positive strain in the mesh 2. Similarly, if a compression force is applied to the auxetic mesh 2 parallel to the x axis, the mesh 2 is compressed in both in directions parallel to the x-axis and the y-axis. Thus, a uniaxial compression force applied to the auxetic mesh 2 causes biaxial negative strain in the mesh 2.

In the non-limiting embodiment shown in the Figures, the auxetic mesh 2 comprises a re-entrant honeycomb structure. As such, the tensile behaviour of the auxetic mesh 2 may be substantially linear. That is, strain of the mesh 2 increases substantially linearly with a force applied to the mesh 2. The re-entrant honeycomb structure of the auxetic mesh 2 is shown in FIG. 2a and a unit cell 3 of this structure is shown in FIG. 2b.

As shown in FIG. 2b, a unit cell 3 of the re-entrant honeycomb structure comprises two vertical struts 4 having a height h and a thickness tₕ and four diagonal struts 5 having a length l and a thickness tₗ. In FIG. 2, the auxetic mesh 2 is arranged so that the vertical struts 4 are parallel to the x-axis. Each diagonal strut 5 extends from a vertical strut 4 at an angle. This angle may be defined by the re-entrant angle θ shown in FIG. 2. The re-entrant angle θ describes 90° minus the angle between a vertical strut 4 and a diagonal strut 5 when the mesh is not subjected to an external force.

FIG. 2 shows the embodiment of the auxetic mesh 2 when the mesh 2 is not subjected to an external force. As a force is applied to the auxetic mesh 2, for example in the x-direction, the angle between the diagonal struts 5 and the vertical struts 4 changes. The angle may change until the unit cell 3 is substantially a quadrilateral in shape (i.e. four diagonal struts 5 are substantially parallel to each another).

The dimensions of the vertical struts 4 and the diagonal struts 5, and the re-entrant angle θ may be selected to achieve a desired stiffness in the auxetic mesh 2. For example, reducing the length of the vertical strut reduces the tensile modulus of the mesh. In certain embodiments, the auxetic mesh 2 may comprise a Poisson's ratio from about −0.7 to about −1.0. For example, the auxetic mesh 2 may comprise a Poisson's ratio of about −0.7, about −0.8, about −0.9, or about −1.0.

In certain non-limiting embodiments, the height of the vertical struts 4 may be from approximately 4.5 to 7.5 mm and the length of the diagonal struts 5 may be from approximately 2.0 to 3.5 mm. When the auxetic mesh 2 is not subjected to an external forces, the re-entrant angle θ may be approximately 30°. The thickness of the vertical struts 4 may be 1.0 mm and the thickness of the diagonal struts 5 may be approximately 1.0 mm. The mesh 2 also comprises a mesh thickness in a direction along the z-axis. In certain non-limiting embodiments, the mesh thickness may be approximately 0.5 to 0.6 mm.

The auxetic mesh 2 may be formed from any material suitable for use in a cell culturing apparatus 1. In certain embodiments, the auxetic mesh 2 may comprise a polymer or a metal. Suitably the auxetic mesh comprise a polymer. Suitably the polymer may be selected from the group consisting of, for example, polypropylene, poly-lactic acid, polystyrene, Teflon®, polycarbonate, polyester, or acrylate. Suitably the auxetic mesh is formed from poly-lactic acid.

As shown in the embodiment in FIG. 1, the cell culturing apparatus 1 comprises a substrate 6 for culturing cells. The substrate 6 is attached to the auxetic mesh 2 so that a change in shape of the mesh 2 causes a corresponding change in the substrate 6.

As described above, if a tension force is applied to stretch the auxetic mesh 2 in a direction parallel to the x-axis, the mesh 2 stretches in both the x and y directions. The substrate 6 is attached to the auxetic mesh 2 so that as the mesh 2 stretches in response to an applied force the substrate 6 stretches too. Strain is transferred from the auxetic mesh 2 to the substrate 6. Thus, in response to a uniaxial tension force being applied to the auxetic mesh 2, biaxial positive strain occurs in the auxetic mesh 2 and in the substrate 6. Similarly, if a uniaxial compression force is applied to the auxetic 2, biaxial negative strain would occur in both the auxetic mesh 2 and in the substrate 6. The substrate 6 is therefore attached to the auxetic mesh such that a change in a biaxial strain in the substrate occurs by the application of a uniaxial force to the auxetic mesh. The biaxial strain induced in the substrate 6 by the auxetic mesh may be used to replicate strain in a natural environment of a cell. The stiffness of the auxetic mesh 2 may be equal to or higher than that of the substrate 6, so that the mechanical behaviour of the substrate 6 is completely controlled by the auxetic mesh.

The cell culturing apparatus 1 may be configured such that when a uniaxial force is applied to the auxetic mesh 2, each point within the substrate 6 is subjected to a biaxial strain. Beneficially, this may ensure that individual cultured cells within the substrate 6 experience a biaxial strain. The cell culture apparatus 1 may therefore enable uniaxial stress to be translated to biaxial strain on a cellular level.

As shown in the embodiment of FIG. 1, the substrate 6 extends in each of the x, y and z directions. That is, the substrate 6 is three-dimensional (3D). Therefore, in response to a change in biaxial strain occurring in the x and y directions of the substrate 6, the stain in the z-direction of the substrate may also change. For example, if the substrate 6 is stretched in both the x and y directions, the substrate 6 may consequently compress in the z direction. As such, if a force applied to the auxetic mesh 2 induces a positive strain in the x and y directions of the mesh 2 and the substrate 6, a negative strain will be induced in the z direction of the substrate 6. Similarly, if a force applied to the auxetic mesh 2 induces a negative strain in the x and y directions of the mesh 2 and the substrate 6, a positive strain will be induced in the z direction of the substrate 6.

In the embodiments shown in the Figures, the auxetic mesh 2 has a re-entrant honeycomb structure. When stretched, deformation in the auxetic mesh 2 is primarily a beam bending which minimises the creation of complex twisting and shearing of the attached substrate 6. The strain induced in both the x and y directions in the substrate 6 is linearly proportional to the strain on the auxetic mesh 2. For example, if a force is applied in the x direction in the auxetic mesh 2, the strain in the x and y directions in the substrate 6 is linearly proportional to the strain in the x direction of the auxetic mesh 2. The Poisson's ratio of a re-entrant honeycomb structure is also predictable. Thus, the combination of a re-entrant honeycomb structure with the substrate 6 is advantageous for inducing strain in a cell culturing apparatus 1 because the strain induced in the substrate 6 is straightforward to predict. As such, the auxetic mesh 6 and the substrate 6 may be used to reliably and accurately of replicate the strain in the substrate 6 that would be present in a natural cell environment.

As shown in the embodiment of FIG. 1, the auxetic mesh 2 may be embedded in the substrate 6. Thus, the mesh 2 extends through the substrate 6. Embedding the auxetic mesh 2 in in the substrate 6 may ensure that the biaxial strain field of the mesh is distributed evenly to the substrate and that a predictable and uniform resultant strain field is produced therein. In contrast, known arrays that stretch the substrate layer using a small number of pins that puncture the substrate can produce an uneven strain field within the substrate.

The mesh 2 may be embedded in the substrate 6 such that the substrate 6 is substantially symmetrical on either side of the plane of the auxetic mesh 2. Passing the mesh through the centre of the substrate 6 in this way may help provide a uniform magnitude of strain through the substrate 6. However, the invention is not limited to the arrangement shown in FIG. 1. The substrate may be attached to the auxetic mesh 2 by any suitable means and/or in a different location. In certain embodiments, the mesh 2 may be bonded directly to the substrate 6. Additionally or alternatively, in certain embodiments, the mesh may be attached to a surface of the substrate 6.

The auxetic mesh 2 may extend beyond at least two opposing surfaces of the substrate 6. This may improve the ease of applying a force to the auxetic mesh 2 without disturbing or disrupting the substrate 6. In the embodiment of FIG. 1, the auxetic mesh 2 has a greater area in the x-y plane than the substrate. 6 Thus, the auxetic mesh 2 extends beyond the surfaces of the substrate 6 in both the positive and negative x and y directions. The substrate 6 may be positioned centrally on the mesh 2 to help provide a uniform magnitude of strain through the substrate 6.

Different types and shapes of substrates 6 for culturing cells may be attached to the auxetic mesh 2 for culturing cells to be used in the cell culturing apparatus 1. The cell culturing apparatus 1 is not limited to a particular type or shape of substrate 6. The substrate 6 may be selected to replicate a physiologically situation.

In certain embodiments, the substrate 6 may comprise a three-dimensional (3D) cell culture substrate or a two-dimensional (2D) cell substrate.

The 3D cell culture substrate may support 3D culture of the cells. By 3D culture it is meant that the cells are able to adopt their natural 3D morphology and distribution within the culture material. That is, the cells are not limited to growing in a single layer, as is the case in 2D cell culture. The 3D cell culture substrate provides a 3D support within which the cells are held such that the natural 3D morphology of the cells is maintained and such that the natural 3D distribution of cells is supported. The cells can proliferate in three dimensions within the 3D cell culture substrate. The strain field of the cell culture assembly 1 is almost entirely defined by the auxetic mesh 2. As such, the strain experienced by the cells is largely independent from the structure of the substrate 6. The substrate 6 can therefore be optimised for other cell culture requirements (e.g., pore connectivity and 3D cell distribution) without affecting the strain imposed on the cells. Beneficially, the strain field experienced by the cells may therefore be more predictable, being consistent for various different substrates each adapted for their physiological applications.

The substrate (such as a 3D cell culture substrate or a 2D cell culture substrate) may be formed from a gel.

The 3D cell culture substrate (for example gel) may be porous. It will be appreciated that the pores shall be of a sufficient size to allow cell infiltration to the pores. The skilled person will be able to determined suitable pore size depending on the type of cells to be grown on the substrate. Suitably, the pores may be between about 25-500 μm, or between about 100-300 μm, or between about 150-250 μm. The 3D cell culture substrate may comprise a porosity of over 5%, over 10%, over 20%, over 30%, over 40%, over 50%, over 60%, over 70%, over 80%, or over 90%.

In an embodiment where the substrate is a gel, the gel may be a hydrogel. Alternatively or additionally, the gel may comprise, for example, Matrigel™, HydroMatrix™ Peptide Hydrogel, MaxGel™ Human ECM, Hystem® Stem Cell Culture, and Geltrex®.

In certain embodiments, the substrate may comprise a protein and/or a polysaccharide. Suitably, the protein may be an extracellular matrix protein. Suitably the extracellular matrix protein may be selected from the group consisting of collagen elastin, laminin, and fibronectin. Details of how to prepare an a substrate comprising collagen are provided in the Examples section of the present disclosure.

In certain embodiments, the substrate 6 may comprises a porous structure. In such embodiments, the application of a force to the auxetic mesh 2 causes an alternation in the pore geometry due to the change in strain in the substrate 6. The alteration of pore geometry may be directly correlated with the force applied to the auxetic mesh 2.

The substrate may be crosslinked.

In certain embodiments, the substrate may comprise cells seeded thereon and/or therein. In this context, by “therein” it is meant that the cells are within the substrate. Cells may be said to be within the substrate, for example they are fully or partially encapsulated by the substrate. It will be appreciated that full or partial encapsulation of the cells is particularly relevant in an embodiment where the substrate is a gel. Additionally or alternatively, it can be said that the cells are within the substrate when they are located fully or partially in the pores of the substrate (i.e. on the surface of the 3D cell growth material that defines the pores of the scaffold). By the term “thereon” it is meant that the cells form a layer (or sheet) on the surface of the substrate. The layer may be a monolayer or multilayer. A multilayer may for example have two, three, four, five, six, seven, eight, nine, ten or more layers of cells.

The cells on and/or in the substrate may be the same or different types of cells. The cells may form a tissue and/or an organoid.

In certain embodiments, the substrate may be configured to mimic a physiological environment, wherein the physiological environment is an organ specific environment. Merely by way of example, the organ specific environment may be a lung environment.

The auxetic mesh 2 may not comprise cells seeded therein or thereon. Cells attached to the auxetic mesh 2 itself would not experience biaxial strain, instead experiencing uniaxial strain as the struts are strained along their length.

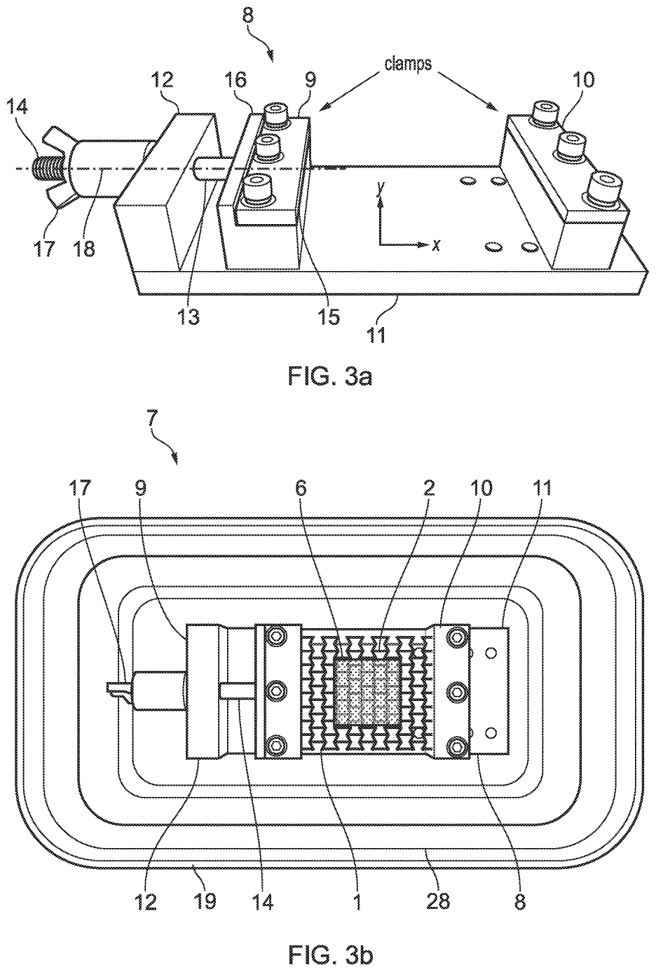

The cell culturing apparatus 1 may be used in an assembly 7 shown in FIG. 3 to culture cells. The assembly 7 includes a clamping device 8 comprising two clamps: a first clamp 9 and a second clamp 10. The clamps 9, 10 are configured to hold the auxetic mesh 2 so that the cell culturing apparatus 1 extends between the first and second clamps 9, 10.

In the assembly 7, the clamps 9, 10 are moveable relative to each other so that a force can be applied to the auxetic mesh 2. In the embodiment of FIG. 3, the clamping device 8 comprises a base 11. The first clamp 9 is moveable secured to the base 11. The second clamp 10 is fixedly secured to the base 11. As such, the first clamp 9 is moveable relative to the second clamp 10.

The first clamp 9 may be engaged threadedly with the base 11 to moveable secure the first clamp 9 to the base 11. As shown in FIG. 3, the base 11 comprises an upstanding shoulder 12 in which an aperture 13 is formed. The first clamp 9 comprises a threaded shaft 14. The threaded shaft 14 is configured to pass through the aperture 13. In the embodiment shown in the Figures, the first clamp 9 is configured such that the cell culturing apparatus 1, when clamped, extends away from a first surface 15 of the first clamp 9 and the threaded shaft 14 extends from a second opposing surface 16 of the first clamp 9.

When assembled, the threaded shaft 14 passes through the aperture 13 in the base 11. A nut 17 is arranged to engage the threaded shaft 14 on the side of the shoulder 12 away from the second surface 16 of the first clamp 9. In the embodiment shown in the Figures, the nut 17 comprises winged nut. The nut 17 may be threaded along the threaded shaft 14 so as to enable the position of the first clamp 9 change relative to the base 11 and the second clamp 10. The first clamp 9 moves in a direction parallel to a longitudinal axis 18 of the aperture 13 and threaded shaft 14. In the embodiment shown in FIG. 3, the longitudinal axis 18 is parallel to the x axis and perpendicular to the y axis.

The assembly 7 includes a bath 19 configured to receive the clamping device 8. The bath 19 may be configured to receive a medium 29 for culturing cells. The bath 19 may be sufficiently deep such that during use the substrate 6 on the cell culturing apparatus 1 may be submerged in a culture medium.

In use, the cell culturing apparatus 1 may be in the clamping device 8. In the embodiment shown in FIG. 3b, the clamps 9, 10 are shown as holding opposing ends of the auxetic mesh 2. The substrate 6 is positioned between the clamps 9, 10. In the embodiment shown in FIG. 3b, the cell culturing apparatus 1 is held in the clamping device 8 so that the verticals struts 4 of the auxetic mesh 2 extend parallel to the x axis i.e. from the first clamp 9 to the second clamp 10. However, the cell culturing apparatus 1 could be held in the clamping device 8 the verticals struts 4 of the auxetic mesh 2 extend parallel to the y axis (i.e. perpendicular to the direction of the first clamp 9 from the second clamp 10). To apply a force to the cell culturing apparatus 1, the nut 17 may be threaded along the threaded shaft 14 to move the first clamp 9 relative to the second clamp 10. In the embodiment in FIG. 3, the position of the nut 17 may be changed to apply or change a uniaxial force to the auxetic mesh 2 parallel to the x axis. As described above, the uniaxial force creates a biaxial strain in the auxetic mesh 2 which in turn induces a corresponding biaxial strain in the substrate 6. As such, the cell culturing apparatus 2 may be used to replicate the natural environment of cells. The clamping device 8 and cell culturing device may be placed in the bath 19. The bath 19 may comprises a culture medium so that the substrate 6 may be disposed in the culture medium. The cell culturing apparatus 1 and substrate 6 may then be maintained under conditions suitable for culturing the cells. As such, cells can be formed within and/or on the substrate 6 as the substrate 6 is subjected to biaxial strain. The term “culture medium” refers to a medium for maintaining a tissue or cell population, or culturing a cell population containing nutrients that maintain cell viability and/or support proliferation. The skilled person will appreciate that the type of culture medium used may be depend upon the type of cells cultured.

During use, the force applied to the auxetic mesh 2 may comprise a physiologically-mimetic force. That is, the force may be selected to replicate physiologically straining situations which occur to biological cells, tissues and organoids. This may be achieved by the force being constant or varying with time, for example, the force may be cyclic. Additionally or alternatively, the magnitude of the force may be selected to replicate physiologically straining situations.

The cell culturing apparatus 1 is not limited to use with the assembly 7 shown in FIG. 3. The cell culturing apparatus 1 may be used in any assembly suitable for holding the cell culturing apparatus 1 and applying a force to the auxetic mesh 2.

The cell culturing apparatus 1 provides a low cost and easy to produce apparatus which enables biaxial strain to be applied a substrate thereby helping replication of a natural environment of cells. The apparatus 1 is also versatile as it can be tuned to replicate physiologically situations by adapting the geometry of the mesh 2 and/or characteristics of the substrate 6. For example, the dimensions of the vertical struts 4 and the diagonal struts 5, and the re-entrant angle may be selected to achieve a desired Poisson's ratio for the auxetic mesh 2 thereby enabling different physiologically situations to be replicated. As such, by changing the dimensions of the vertical struts 4 and the diagonal struts 5, and the re-entrant angle meshes with different Poisson's ratios may be provided. The cell culturing apparatus 1 is not limited to a particular cell culturing substrate or culturing a single cell layer. Rather, numerous different types of cell culturing substrates 6 can be attached to the mesh 2. Potential applications of the cell culturing apparatus 1 range from academic laboratory equipment for advanced tissue engineering studies to next generation pharmaceutical drug testing platforms.

The cell culturing apparatus 1 may be manufactured by a method comprising providing an auxetic mesh 2. In certain embodiments, providing an auxetic mesh 2 may comprises 3D-printing the auxetic mesh 2. For example, the auxetic mesh 2 may be 3D printed using a poly-lactide filament.

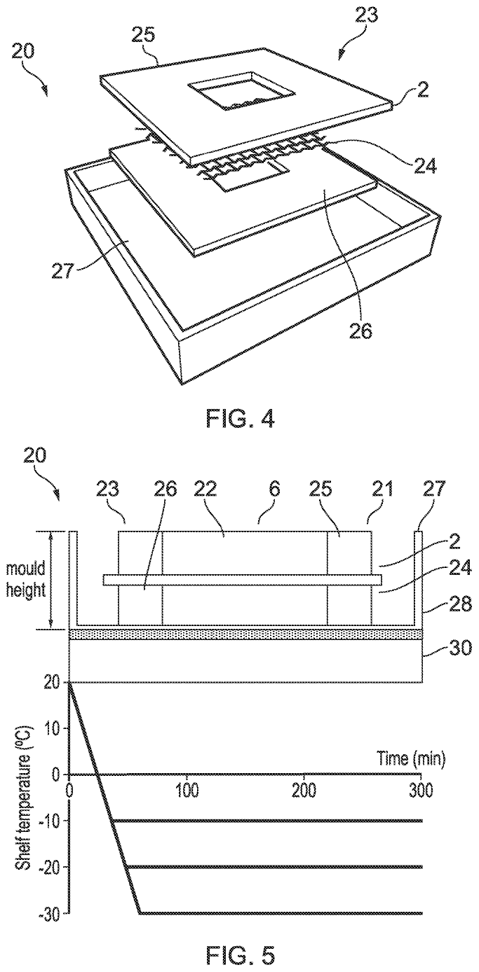

The method comprises associating the auxetic mesh 2 with a substrate 6. The apparatus 20 shown in FIGS. 4 and 5 may be used to associate the auxetic mesh 2 with a substrate 6. The apparatus 20 comprises a mould 21 in which the auxetic mesh 2 may be placed. The mould 21 comprises a cavity 22 for receiving a substrate medium. In certain embodiments, the mould 21 may be formed from silicone. Optionally, the substrate medium may comprise cells.

The mould 21 comprises a upper section 23 and a lower section 24. Each of the upper and lower sections 23, 24 define a channel 25, 26 extending therethrough for receiving the substrate medium. When the upper and lower sections 23, 24, are assembled, the channel 25 in the upper section 23 is aligned with the channel 26 in the lower section 24 so as to form the cavity 22 for receiving the substrate.

In certain embodiments to form the cell culturing apparatus 1, the auxetic mesh 2 may be placed between the upper section 23 and the lower section 24 of the mould 21. When positioned within the mould 21, the mesh 2 extends across the cavity 22 so that a part of the mesh 2 is suspended within the mould 21. The mesh 2 may be positioned such that the centre of the mesh 2 coincides with the centre of the cavity 22. The mould 21 may then be placed within a weighing boat 27. In certain embodiments, the weighing boat 27 may be formed from polystyrene. One or more weights (not shown) may be placed on the upper section 23 of the mould 21 to secure the upper section 23, lower section 24 and mesh together 2. The substrate 6 is then formed in the cavity 22 of the mould 21. In certain embodiments, the substrate 6 may be formed by pouring a substrate medium, such as a slurry or fluid, into the cavity 22 of the assembled mould 21. The substrate medium may passes through the mesh 2 to fill the cavity as shown in FIG. 5.

In an alternative embodiment to form the cell culturing apparatus 1, the lower section 24 of the mould 21 may be placed in the weighing boat 27. The channel 26 of the lower section 24 may then be filled by a substrate medium. Once the channel 26 has been filed, the auxetic mesh 2 may be placed on the lower section 24 and then the upper section 23 may be placed onto top of the mesh 2 and the lower section 24. In the same way as described above, the mesh 2 extends across the cavity 22 so that a part of the mesh 2 is suspended within the mould 21. One or more weights (not shown) may be placed on the upper section 23 of the mould 21 to secure the upper section 23, lower section 24 and mesh together 2. The channel 25 of the upper section 23 may then be filled by a substrate medium so that the substrate medium fills the cavity 22.

The apparatus 20 may be degassed in vacuum to ensure the mesh 2 is fully immersed in the substrate medium. The apparatus 20 may then be freeze dried to form the substrate 6 from the substrate medium. In certain embodiments, the apparatus 20 may be placed on a cold shelf 30 in a freeze drier. The substrate medium may then be freeze dried according to a protocol, for example, the temperature of the cold shelf 30 may be lowered to a final freezing temperature at a fixed rate. In certain embodiments, an air gap 28 may be used to separate the weighting bath 27 from the shelf 30 of the freeze drier to provide air insulation. In certain embodiments, the air gap may be provided by placing the weighing bath on corrugated cardboard.

The shape and size of the resulting substrate 6 is defined by the cavity 22 in the mould 21. In the non-limiting embodiment shown in FIGS. 4 and 5, the upper section 23 and the lower section 24 are identical. However, the mould 21 may take an alternative shape. The cavity 22, and channels 25, 26 in the upper and lower sections 23, 24 of the mould 21, may be chosen to provide any desired substrate geometry.

Once the substrate 6 has formed through freeze-drying, the substrate 6 may be cross linked using any conventional process. An exemplary method for cross-linking in provided in the Examples section of the present application. The mesh 2 and substrate 6 may then be cut or shaped to provide the cell culturing apparatus 1. Finally, cells may be applied to the substrate 6 prior to use of the cell culturing apparatus 1. For example, the substrate 6 may be seeded with cells. The cells may be seeded on or within the substrate 6. The cell culturing apparatus 1 may then be used for culturing cells, for example, in the assembly 7 of FIG. 3.

In an alternative embodiment, the cells may be combined with a substrate medium prior to the substrate medium being poured into a mould 21 and forming a substrate 6. It will be appreciated that in such an embodiment the cells may be partially or fully encapsulated by the substrate.

In certain embodiments, the substrate 6 may be formed by pouring a substrate medium, such as a slurry or fluid, into the cavity 22 of the assembled mould 21. The substrate medium may passes through the mesh 2 to fill the cavity as shown in FIG. 5.

The skilled person would understand that various modifications can be made to the above described embodiments.

For example, the invention is not limited to an auxetic mesh 2 having a re-entrant honeycomb structure, alternative mesh structures may be used. For example, any mesh having a negative Poisson's ratio may be used. That is, any auxetic metamaterial sheet or auxetic mesh may be used. For example in certain embodiments, the auxetic mesh may comprise a repetition of S-shaped unit cells, a repetition of C-shaped unit cells and a repetition of cubic chiral unit cells. In certain embodiments, the auxetic mesh 2 comprises a Poisson's ratio from −0.7 to −1.0. Preferably, the auxetic mesh has a Poisson's ratio which is predictable.

The auxetic mesh 2 is not limited to a 2D auxetic mesh. In certain embodiments, the auxetic mesh 2 may comprise a three-dimensional 3D auxetic mesh. In such embodiments, the substrate is attached to the 3D auxetic mesh such that a change in triaxial strain in the substrate occurs by the application of a uniaxial force to the auxetic mesh. That is, the substrate is attached to the 3D auxetic mesh such that a change in a triaxial strain in the substrate occurs by a corresponding change in triaxial strain in the auxetic mesh due to the application of a uniaxial force to the 3D auxetic mesh. For example, in response to a uniaxial tension force being applied to the 3D auxetic mesh, triaxial positive strain occurs in the 3D auxetic mesh and in the substrate. Similarly, if a uniaxial compression force is applied to the auxetic, triaxial negative strain would occur in both the auxetic mesh and in the substrate. In the embodiment of FIG. 1, the substrate 6 is substantially cuboid in shape. However, the substrate 6 is not limited to this shape but make take any alternative shape.

Furthermore, the cell culturing apparatus 1 is not limited to use with the assembly 7 shown in FIG. 3. The cell culturing apparatus 1 may be used in any assembly suitable for holding the cell culturing apparatus 1 and applying a force to the auxetic mesh 2.

Herein is also provided, use of an auxetic mesh for cell culture. Suitably the auxetic mesh may comprise a Poisson's ratio of −0.7 to −1.0. Suitably the auxetic mesh may comprise a re-entrant honeycomb structure. Suitably, the mesh may be formed from poly-lactic acid.

### EXAMPLES

### Materials and Methods

Unless otherwise specified, all chemical reagents were purchased from Sigma Aldrich.

In the following examples, the cell culturing apparatus 1 is composed of an auxetic mesh 2 embedded in a substrate 6 for culturing cells. In this example, the substrate is a collagen tissue engineering scaffold. In this study, a re-entrant honeycomb 2D mesh was used as the auxetic mesh. The mesh was 3D printed using commercial poly-lactide (PLA) filament. The cell substrate was chosen to be ice-templated collagen scaffolds.

### Auxetic Mesh Production

The re-entrant auxetic meshes were printed using a Prusa i3 MK2 3D printer. The nozzle temperature was 215° C., and the bed temperature was 55° C. To ensure printing layer consistency and flatness, a sacrificial polyvinyl acetate (PVA) layer was printed first, and the PLA layers were printed on top of the PVA layer. The PVA layer was then removed after printing. [1] FIG. 2b demonstrates the geometry of the mesh unit cell used in the study. Meshes containing 8×11 unit cells were printed with 4 different unit cell geometries as summarised in table 1. The re-entrant angle (θ) was kept at −30° for all meshes, strut widths (tₕ and tₗ) were kept the at 1 mm, and the mesh thickness (b) in direction a direction perpendicular to the x-y plane was kept at 0.57 mm. In Table 1, h is the vertical struts height,/is the diagonal strut length and t is the thickness of the vertical and diagonal struts.

TABLE 1
Summary of the geometry parameters of the mesh printed.

| Index | h (mm) | / (mm) | t (mm) | b (mm) | h/l | t/h | t/ |
|---|---|---|---|---|---|---|---|
| 1 | 4.5 | 2.2 | 1 | 0.57 | 2.045 | 0.222 | 0.455 |
| 2 | 5.5 | 2.6 | 1 | 0.57 | 2.115 | 0.182 | 0.385 |
| 3 | 6.4 | 3.1 | 1 | 0.57 | 2.065 | 0.156 | 0.323 |
| 4 | 7.3 | 3.5 | 1 | 0.57 | 2.086 | 0.137 | 0.286 |

### Slurry Preparation

To produce a collagen scaffold (i.e. the substrate 6), 1 wt. % collagen slurry was prepared by homogenising Fibrillar bovine dermal type I collagen (Devro medical) hydrated in 0.05 M acetic acid. The homogenisation was carried out in a Waring commercial blender for 2×2 min at 18000 rpm and then 22000 rpm. The slurry was allowed to rest for 2 min in between to avoid denaturing. Homogenised collagen slurry was then degassed by centrifuging at 2500 rpm for 5 min and then by vacuum at 60 Torr. Degassing was considered complete when no more gas bubbles appeared in the slurry under vacuum

### Freeze-Drying

The assembly 20 shown in FIGS. 4 and 5 was used to produce a cell culturing apparatus 1. The auxetic mesh 2 with a vertical strut length of 6.4 mm was sandwiched between upper and lower silicone moulds 23, 24 of dimensions 3 cm×3 cm with various heights, and they were placed on a polystyrene weighing boat 27. In the study, the upper and lower silicone moulds 23, 24 had the same height as each other, and the overall mould 21 height was recorded as twice the single mould height and varied between 3 mm and 9 mm. The mould height is illustrated in FIG. 4. The upper and lower moulds 23, 24 were aligned, and the mesh 2 was placed so that the freeze-dried scaffold would be at the centre of the mesh 2. After filling the mould 21 with the prepared collagen slurry, the whole assembly was freeze-dried according to a set protocol in a VirTis Advantage freeze-drier. The temperature of the cold shelf 30 was lowered to a final freezing temperature at a cooling rate of −0.83° C. min⁻¹ and held for 4 hours. The final freezing temperature was varied between −10° C. and −30° C. An air gap 28 could be introduced in the form of a corrugated cardboard of thickness 2.5 mm so as to separate the collagen slurry from the cold shelf with air insulation. Ice sublimation was then facilitated at a vacuum of 80 mTorr for 20 h. The assembly 20 in the freeze drier is illustrated in FIG. 5.

### Crosslinking

The collagen scaffolds were then cross-linked using 1-ethyl-3-(3-dimethylaminopropyl) carbodiimide (EDC) and N-hydroxysuccinimide (NHS) dissolved in 75% ethanol. The molar ratio of EDC:NHS:COO⁻ was set to be 5:2:10. The samples were completely immersed in the cross-linking solution and placed on a shaker at 100 rpm for 2 hrs. The scaffolds were then washed by completing immersion in deionised water on a shaker for 4×30 min at 100 rpm. Water was changed between washes. Where a dry scaffold was necessary, the device was then freeze-dried according to the same protocol.

### Collagen Scaffold Substrate Porous Architecture Characterisation

Samples comprising the whole thickness of the scaffolds and the embedded mesh were cut from the cell culturing apparatus and were imaged using a SkyScan 1172 micro-CT. The X-ray source was operating at 25 kV, 138 μA, and the exposure time to the camera was 1900 ms. No filter was used. The achieved pixel size was 2.97 μm. Reconstruction of the raw micro-CT data was carried out by NRecon (Bruker) to produce a 3D dataset. 1 mm³ cubic volumes of interest (VOIs) were taken from the reconstructed dataset at positions immediately above and below the auxetic mesh. The position below the mesh was adjacent to the cold shelf of the freeze drier during production. From the VOIs, several structural parameters of the porous structure were extracted using CTAnalyser (Bruker) including pore size, pore anisotropy, and percolation diameter. The percolation diameter is a mathematically extrapolated parameter that reflects the maximum size of a particle that can travel through an infinite interconnected network. To compute the percolation diameters, voxel clusters of defined sizes (d), 6 μm to 264 μm, were probed across the VOIs. For each voxel cluster, the longest distance the probe can access (L) before being blocked by the porous network due to limited pore interconnection was recorded. d was then plotted against L^{-1/0.88}, and the y-intercept (dc) was obtained as the percolation diameter, according to percolation theory for 3D systems [2]:

$$L = {{L}_{0} ( d - {d}_{c} )}^{- 0.88} \qquad Equation 1$$

where L₀ is a constant. To obtain accurate percolation diameters free from the effect of edge/centre artefacts that could penetrate the entire height of the VOIs, the method developed by Nair et al was used [3]. Briefly, the extrapolation process was iterated over progressively smaller VOIs with smaller basal surface area to exclude the artefacts. After 20 iterations, the plateau percolation diameter value plotted against the decreasing VOI sizes was used as the accurate percolation diameter for each VOI.

### Mechanical Properties Characterisation

### Collagen Scaffolds

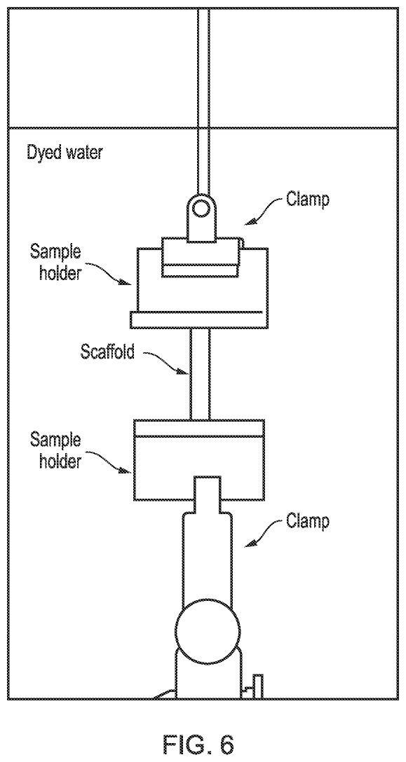

Collagen scaffolds of various pore sizes were produced as described above. The same freeze-dry mould and protocol were followed, except in this case, no auxetic mesh was embedded. Crosslinked dry scaffolds were cut into samples of dimensions 3 mm×10 mm×5 mm (length×width×height). The samples were adhered to 3D printed PLA sample holders using ethyl cyanoacrylate and immersed in a water chamber for tensile testing in hydrated conditions. The tensile testing was performed on a Tinius Olsen 1ST testing machine at a strain rate of 0.004 s⁻¹ with a 1 kN load cell. The wet condition tensile testing setup is shown in FIG. 6. As shown in FIG. 6, the set up comprises two clamps which each hold a sample holder. Opposing ends of a hydrated collagen scaffold are held by each sample holder. The scaffold is submerged in water. The clamps are movable so as to apply forces across the scaffold. Tensile moduli were obtained by linear regression from 0.05 to 0.10 strain on the stress-strain curves.

### Auxetic Mesh

Tensile testing was also performed on 3D printed re-entrant honeycomb meshes of various geometries produced as described above. The tensile testing was performed on a Tinius Olsen 1ST testing machine with a 1 kN load cell. Since the stress-strain curves remained linear shortly before break, tensile moduli were obtained at 0.02 strain.

### Tensile Actuation of Cell Culturing Apparatus

Tensile actuation of the cell culturing apparatus 1 was achieved using the assembly 7 shown in FIG. 3 and described above. Ends of the auxetic mesh 2 were clamped to the clamping device 8, so that the mesh 2 between clamps 9, 10 comprised 11 unit cells of length Lₓ in the x direction and 8 unit cells of length L_{y} in the y direction. The second clamp 10 was fixed, and the first clamp 9 was displaced by controlled distances (ΔLₓ).

### Poisson's Ratio and Induced Strain Characterisation

### Auxetic Mesh

Determination of Poisson's ratio of the auxetic meshes was done using the assembly 7 as described above. Strain along the x direction (ϵ_{x,mesh}) was calculated as

$${∈}_{x , mesh} = \frac{Δ {L}_{x}}{{L}_{x}} \qquad Equation 2$$

After the displacement of the first clamp 9 relative to the second clamp 10, the maximum expansion of the mesh along the y direction (Δ_{L}_{_{y}}_{) was recorded. The measurement was made along the central line of the mesh where maximum expansion was expected. Strain along the y direction (ϵ}_{y,mesh}) was calculated as

$${∈}_{y , mesh} = \frac{Δ {L}_{y}}{{L}_{y}} \qquad Equation 3$$

Poisson's ratio of the meshes of the various dimensions were measured at ϵ_{x, mesh}=0.03 (approximately the linear strain in lung air sacs during normal breathing) and calculated as

$${v}_{mesh} = - \frac{{∈}_{y , mesh}}{{∈}_{x , mesh}} \qquad Equation 4$$

The theoretical in-plane Poisson's ratio of the meshes were calculated according to the analytical solution reported by Whitty et al [4]:

$${υ}_{xy} = \frac{sin θ ( \frac{h}{l} + sin θ ) ( \frac{1}{{K}_{f}} + \frac{1}{{K}_{h}} + \frac{1}{{K}_{s}^{( l )}} )}{[ {cos}^{2} θ ( \frac{1}{{K}_{f}} + \frac{1}{{K}_{h}} ) + \frac{{sin}^{2} θ}{{K}_{s}^{( l )}} + \frac{2}{{K}_{s}^{( h )}} ]} \qquad Equation 5$$

where the force constants, K, are expressed as

$${K}_{f} = {E}_{strut} {b ( \frac{{t}_{l}}{l} )}^{3} \qquad Equation 6$$

$${K}_{h} = {G}_{strut} b ( \frac{{t}_{l}}{l} ) \qquad Equation 7$$

$${K}_{s}^{( l )} = {G}_{strut} b ( \frac{{t}_{l}}{l} ) \qquad Equation 8$$

$${K}_{s}^{( h )} = {E}_{strut} b ( \frac{{t}_{h}}{h} ) \qquad Equation 9$$

and Eₛₜᵣᵤₜ and Gₛₜᵣᵤₜ are the Young's modulus and shear modulus of the strut material of the mesh, respectively. The force constants account for the flexing, hinging, and stretching of the struts during tensile deformation.

### Collagen Scaffold Substrate on Mesh

In a fully hydrated state after cross-linking, the cell culture apparatus 1 was clamped to the clamping device 8 as described above. The chosen mesh geometry had a vertical strut length of 6.4 mm, and other parameters were summarised in Table 1. The clamping device 8 was then immersed in a water bath 19. Changes in scaffold dimensions along the x and y directions (ΔSₓ and ΔS_{y}, directions indicated in FIG. 3) were measured when a tensile strain was applied using the tensile rig at ϵ_{x, mesh}=0.015 and 0.03, and the corresponding induced strains on the scaffolds (ϵ_{x, substrate} and ϵ_{y, substrate}) were calculated.

### Fibroblast Strain Testing

Testing was also performed to assess the effect of straining the auxetic mesh 2 on human dermal fibroblasts. The human dermal fibroblasts were cultured in 3 wt. % collagen gels made from soluble collagen. The collagen gel was implemented as the substrate 6 in the cell culture apparatus 1. The auxetic mesh 2 was embedded in the collagen gel. The cells were cultured in media in an unstrained state for 18 hours before a 5% strain was applied using the auxetic mesh 2. A control group was run in parallel and remained unstrained. Three biological repeats were performed and t-tests were used to assess the statistical significance of the data.

### Finite Element Analysis

Three-dimensional models of a PLA re-entrant honeycomb mesh and a porous collagen scaffold bonded to a PLA re-entrant honeycomb mesh were constructed and simulated using the commercial finite element modelling package COMSOL® Multiphysics.

### Simulation of the Re-Entrant Honeycomb Mesh

To represent the experimental assembly whereby PLA meshes were clamped by the clamping device 8, the meshes were constructed with bounded ends as shown in FIG. 2. In FIG. 2a, h₀ represents the original length of the auxetic mesh in the x direction and w₀ represents the original distance in the y direction between the two red dots 31, 32. The mesh material was chosen to be PLA (E_{PLA}=3.50 GPa, v=0.36), and the bounding end steel (Eₛₜₑₑₗ=3.50 GPa, v=0.33). The minimum finite element size was set to 2 mm. The behavior under tensile strain of a series of bounded meshes with a theoretical Poisson's ratio of −1 were simulated. The meshes contained 10×10 unit cells of various geometry parameters as summarised in Table 2. One of the bounded ends were fixed as the boundary condition and the other end was displaced along the x direction for a distance of Δh. The strain in the x direction was calculated as

$${∈}_{x , sim} = \frac{Δ h}{{h}_{0}} \qquad Equation 10$$

ϵ_{x, sim} was kept at 0.03. The strain in the y direction (ϵ_{y, sim}) was calculated as

$${∈}_{y , sim} = \frac{Δ w}{{w}_{0}} \qquad Equation 11$$

where w₀ is the original distance in the between the red dots 31, 32 illustrated in FIG. 2a, and Δw is the change in mesh length in the y direction after applying strain. The actual Poisson's ratio, vₛᵢₘ, of the simulated meshes were calculated as

$${v}_{sim} = - \frac{{∈}_{y , sim}}{{∈}_{x , sim}} \qquad Equation 12$$

### Simulation of the Cell Culturing Apparatus

The fine pore size of the scaffold in comparison to the mesh cell size allowed the scaffold to be approximated to a uniform block with the properties of a collagen-air composite (E_{col}=14 kPa, v=0.1). Within the simulation, a collagen scaffold of dimensions 71.2 mm×44.8 mm×3 mm was bonded to an auxetic mesh as shown in FIGS. 1 and 3. The scaffold was placed at the centre of the mesh covering exactly 10 unit cells. The element size was selected to be 700 μm such that nodal separation was on the order of a pore size, allowing a sufficient resolution of data points to be extracted. These data points were then joined into random isotropic pores with an approximate size of 500 μm by spatially iterating through the scaffold and applying a 3-dimensional convex hull algorithm. Each pore volume was extracted both prior to and after applying a macroscopic tensile strain of 0.03.

### Statistics

All measurements were repeated for at least three times for different samples or VOIs if relevant. Errors were reported as the standard error of the mean

TABLE 2
Geometry parameters of the simulated re-entrant
honeycomb meshes with theoretical Poisson’s ratio of -1

| Index | tₗ/l | tₕ/ | h/l | tₗ (mm) | l (mm) | tₕ (mm) | h (mm) | b (mm) |
|---|---|---|---|---|---|---|---|---|
| 1 | 0.1563 | 0.7332 | 2.065 | 1.000 | 6.398 | 9.687 | 13.212 | 2.000 |
| 2 | 0.1563 | 0.5703 | 2.072 | 1.000 | 6.398 | 7.56 | 13.257 | 2.000 |
| 3 | 0.134 | 0.3296 | 2.063 | 1.000 | 7.463 | 6.614 | 15.396 | 2.000 |
| 4 | 0.1116 | 0.2592 | 2.051 | 1.000 | 8.961 | 4.764 | 18.378 | 2.000 |
| 5 | 0.1042 | 0.1333 | 2.057 | 1.000 | 9.597 | 2.631 | 19.741 | 2.000 |
| 6 | 0.335 | 0.6666 | 2.379 | 1.000 | 2.985 | 4.734 | 7.101 | 2.000 |
| 7 | 0.3425 | 0.4222 | 2.519 | 1.000 | 3.053 | 3.105 | 7.355 | 2.000 |
| 8 | 0.335 | 0.237 | 2.719 | 1.000 | 1.975 | 1.924 | 8.116 | 2.000 |
| 9 | 0.3276 | 0.1333 | 3.087 | 1.000 | 1.946 | 1.256 | 9.423 | 2.000 |
| 10 | 0.5063 | 0.4962 | 3.103 | 1.000 | 1.975 | 3.041 | 6.129 | 2.000 |
| 11 | 0.5138 | 0.3333 | 3.497 | 1.000 | 1.946 | 2.268 | 6.806 | 2.000 |
| 12 | 0.5063 | 0.1259 | 5.284 | 1.000 | 1.975 | 1.314 | 10.437 | 2.000 |
| 13 | 0.6925 | 0.3259 | 4.825 | 1.000 | 1.444 | 2.271 | 6.968 | 2.000 |
| 14 | 0.6999 | 0.1629 | 7.212 | 1.000 | 1.429 | 1.679 | 10.304 | 2.000 |
| 15 | 0.8414 | 0.1703 | 9.212 | 1.000 | 1.188 | 1.865 | 10.948 | 2.000 |
| 16 | 0.1116 | 0.0519 | 2.122 | 1.000 | 8.961 | 0.987 | 19.014 | 2.000 |

### Results

### Static Porous Architecture of the Collagen Scaffold Substrate

Having an appropriate porous architecture is the first step towards an ideal tissue engineering substrate. Here, it is shown that by changing various parameters during the freeze dry process, a range of physiologically relevant porous architecture can be achieved.

### Effect of Final Freezing Temperature

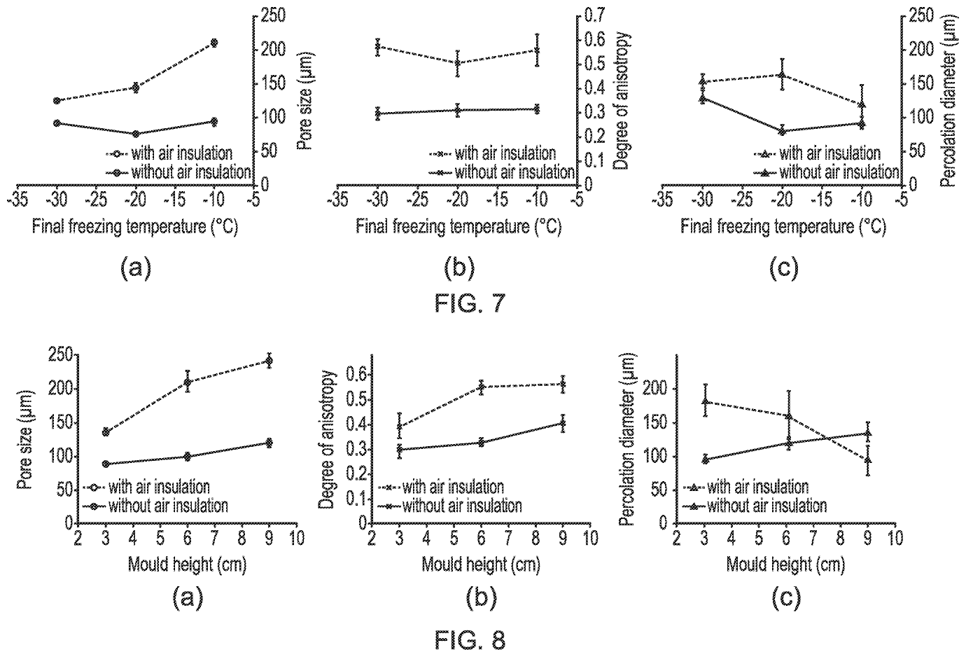

The final freezing temperature (FFT) is the temperature of the freeze drier cold shelf at which freezing occurs. It was one of the dominant factors that determined the pore size of the scaffolds. Increase in FFT was expected to lead to an increase in pore size due to its effect on the heat extraction efficiency of the cold shelf. This effect was more prominently observed for scaffolds produced with air insulation as shown in FIG. 7. By increasing the FFT from −30° C. to −10° C., the pore size of the scaffolds increased from 128 μm to 212 μm. The effect of FFT on the degree of anisotropy (DA; 0=completely isotropic, 1=completely anisotropic) was negligible across the FFTs regardless of the air insulation. Increase in FFT led to a decrease in the percolation diameters of approximately 30 mm from −30° C. to −10° C. with and without air insulation. The relative small change and the slight inconsistency at −20° C. suggested a weak dependency for the percolation diameters.

### Effect of Mould Height

Similar to the effect of FFT, increase in mould height led to an increase in pore size as shown in FIG. 8. For scaffolds made with air insulation, the pore size increased from 132 μm to 238 μm, when the mould height was increased from 3 cm to 9 cm. Mould height also has a consistent effect on the DA, where the scaffolds became marginally more anisotropic with increase in mould height. Clear trends were also observed for the percolation diameters, but opposite effects were seen with and without air insulation. The percolation diameter for scaffolds made with air insulation decrease from 178 μm to 92 μm, but increased from 94 μm to 133 μm without air insulation. Although less predictable, the percolation diameters observed in all conditions (see FIGS. 7 and 8) were considerably larger than the size of commonly used cells during tissue engineering, which suggested that they are ideal for cell infiltration during culturing. For example, lung alveolar progenitor cells have an average size of 18 μm [5], so all scaffolds with a percolation diameter higher than 18 μm should allow improved infiltration.

### Effect of Air Insulation

As mentioned above, air insulation can have a strong and significant effect on the pore architecture. Consistent across the data in FIGS. 7 and 8, removing the air insulation resulted in a systematic decrease in pore sizes. Also any trends observed against FFT and mould height became less prominent without air insulation. Similarly, scaffolds made with air insulation were consistently more isotropic, and their percolation diameters were also lower except for the scaffolds frozen at 9 cm mould height (see FIG. 8). This suggested that, in general, a lower pore size corresponds to a lower percolation diameter. The change in pore size due to change in FFT and mould height may be too small for this phenomenon to be obvious.

### Effect of Mesh

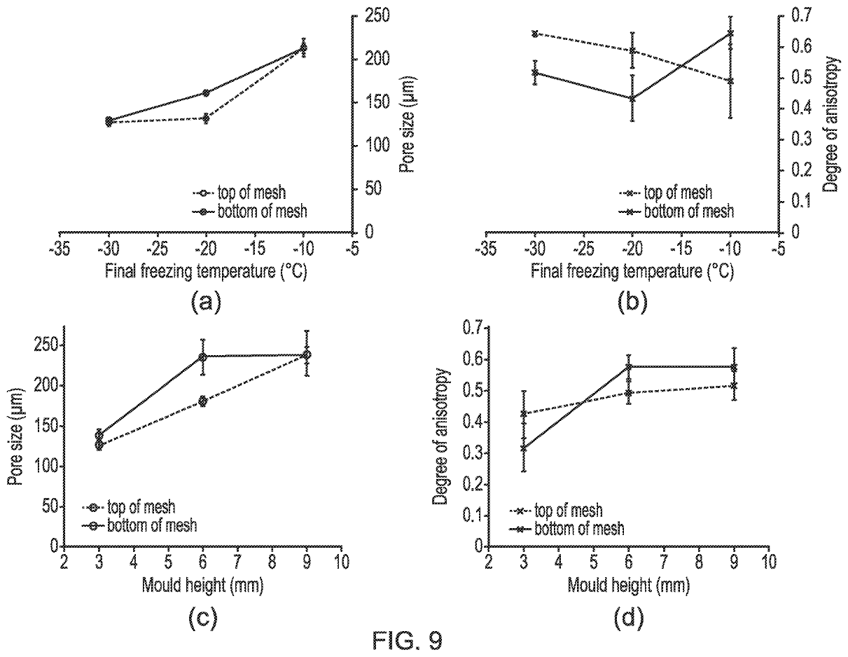

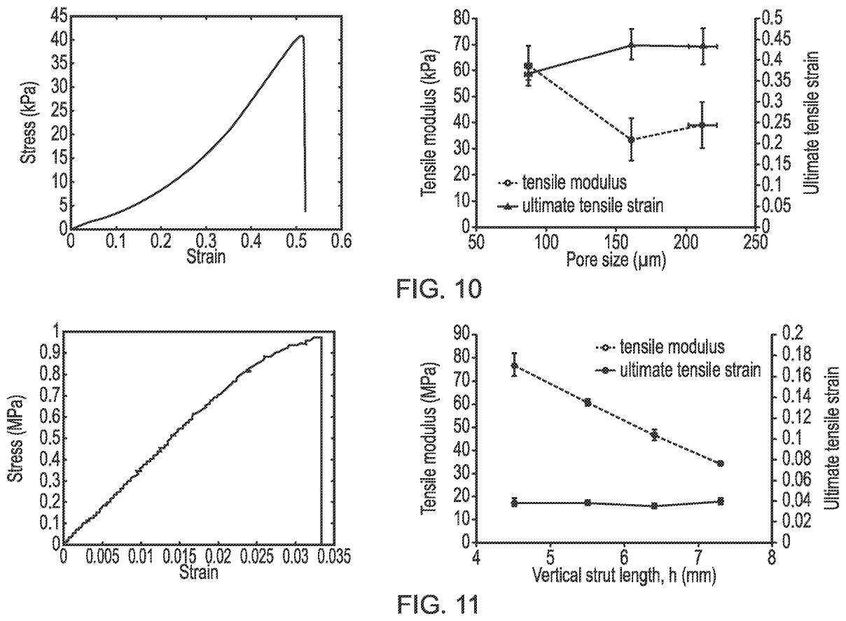

To ensure pore architecture parameter data reported above were not compromised by the embedded mesh in the cell culturing apparatus, pore size and DA were compared above and below the mesh while varying FFT and mould heights as described above. The difference in pore size across FFTs and mould heights were minimal as shown in FIG. 9, and they all followed the same trend. The only noticeable differences observed were for conditions at −20° C. and 6 cm mould height, but the difference was within 50 μm. The difference in DA was also in a tolerable range with the maximum difference of 0.15 observed for samples frozen at −20° C.

### Mechanical Properties

### Collagen Scaffolds

The stress-strain curves of the collagen scaffolds were nonlinear as shown in FIG. 10. Such behaviours are also found in biological tissues such as the lung parenchyma [6]. To simplify the calculations, tensile modulus was obtained from 0.05 to 0.10 strain where the segment of the curve was close to linear. The tensile moduli of the scaffolds decreased with increasing pore sizes as also shown in FIG. 10. The highest modulus of 62 kPa was observed at the lowest pore size of 87 mm. Once the pore size was above 150 mm, the tensile moduli was stabilised at approximately 35 kPa. These values were on the same order of magnitude as the tensile modulus reported for animal lung tissues. At the same strain range, the tensile moduli for rabbit lung was 12.6 kPa and 16 kPa for rat lung measured at a strain rate of 25 min⁻¹ [6]. It is worth noting that human lungs contain much higher collagen content that mentioned species, and, therefore, a higher stiffness would be expected [7], making the collagen scaffolds appropriate for lung tissue engineering. The ductility, represented here as the ultimate tensile strain, showed opposite trend as the stiffness as expected.

### Auxetic Mesh

The tensile behaviour of the auxetic meshes were largely linear as shown in FIG. 11. This means prediction of stress state would be straightforward at various strains, making them ideal for mechanical manipulation of the scaffolds. Increasing the vertical strut length made the meshes less stiff in a linear manner as shown in FIG. 11. The strut width and thickness were kept constant, so the overall material density decreases with the strut length, which explains the behaviour. The stiffness of the meshes were orders of magnitudes higher than the stiffness of the collagen scaffolds, so the mechanical deformation induced on the scaffold substrates in the cell culturing apparatus would be determined completely by the strain states of the mesh. In contrast, the ultimate tensile strain remained unchanged across the lengths as shown in FIG. 11. Based on the ease of stretching, the production throughput, and the yield, the mesh with a vertical strut length of 6.4 mm was chosen.

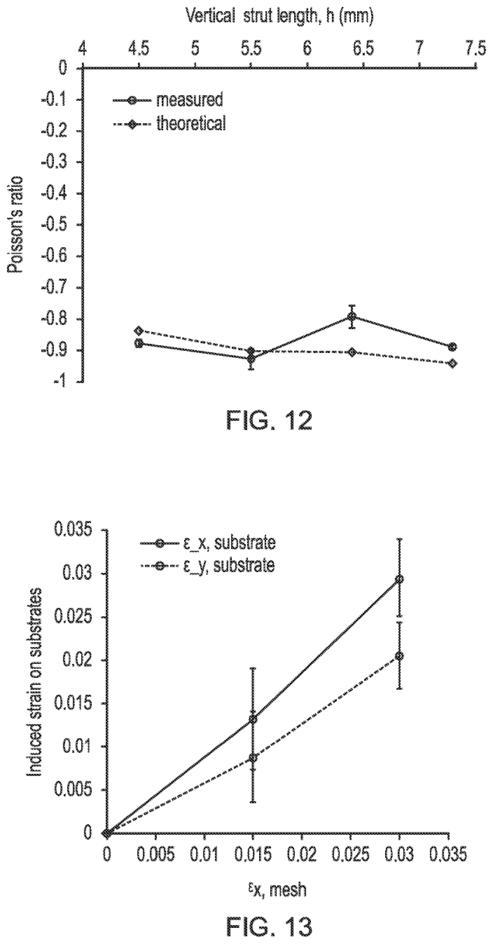

Poisson's ratio of meshes of different geometries were measured and plotted against the vertical strut length, and compared with the theoretical values calculated according to Equation 5. The comparison is shown in FIG. 12. Due to the limiting boundary condition of the clamped mesh, the measured Poisson's ratio was expected to be less negative than the theoretical values. However, this was only observed for meshes with higher vertical strut length. The most positive value was observed for the mesh with a strut length of 6.4 mm at −0.79. Smaller meshes actually displayed more negative ratios than their theoretical counterpart, meaning they behaved more auxetic than expected. The most auxetic mesh with a measure ratio of −0.93 was the one with a strut length of 5.5 cm.

### Cell Culturing Apparatus

The produced cell culturing apparatus was subjected to uniaxial strain on the auxetic mesh (ϵ_{x, mesh}), and the resulting induced strain on the collagen scaffold substrate on the device was measured and plotted against ϵ_{x, mesh} as shown in FIG. 13. The strain induced in both directions in the scaffold substrate (ϵ_{x, substrate} and ϵ_{y, substrate}) increase linearly with ϵ_{x, mesh}. The coefficient of proportionality of ϵ_{x, substrate} was close to unity. The coefficient of ϵ_{y, substrate} was less than 1 (approximately 0.68) as expected, due to the Poisson's ratio of the embedded auxetic mesh. The result demonstrates that induced biaxial straining of the bonded scaffold substrate can be achieved with uniaxial loading on the auxetic mesh.

### Fibroblast Strain Testing Results

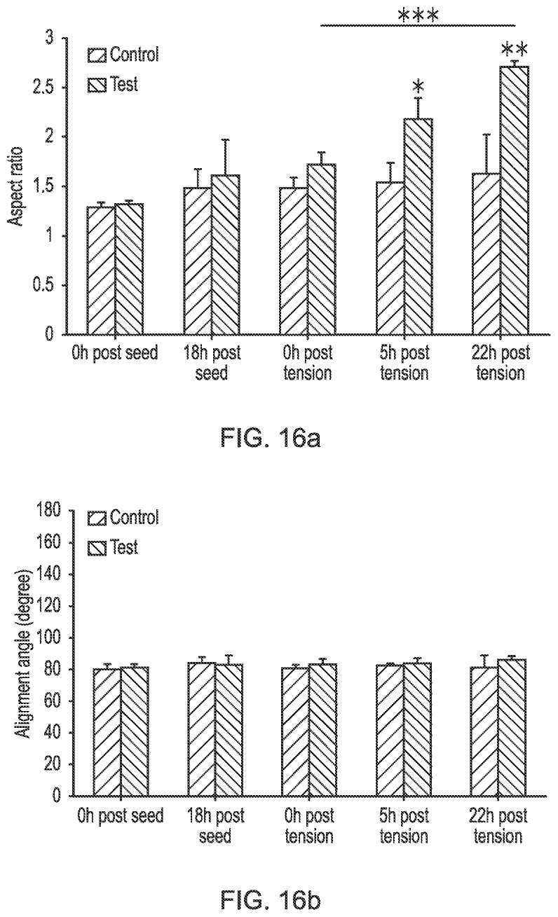

FIG. 16a shows the aspect ratio of human dermal fibroblasts exposed to 5% strain at five timepoints against the aspect ratio of an non-strained control group.

As shown, the aspect ratio of the test group increased over time. This illustrates that cells exposed to the 5% strain became elongated in response to the applied tension. After 5 h of applied strain, the aspect ratio of the cells was significantly higher that that of the control. The aspect ratio of the strained cells increased significantly after 22 h of applied strain.

FIG. 16b shows the averaged alignment angle of the embedded cells at five timepoints during straining. As shown, the test group of cells subjected to the 5% strain remained randomly aligned. No statistically significant difference was observed compared to the control group. Importantly, these results highlight that the cells experienced a biaxial strain resulting in their elongation in all directions.

### Finite Element Stimulation

### Auxetic Mesh

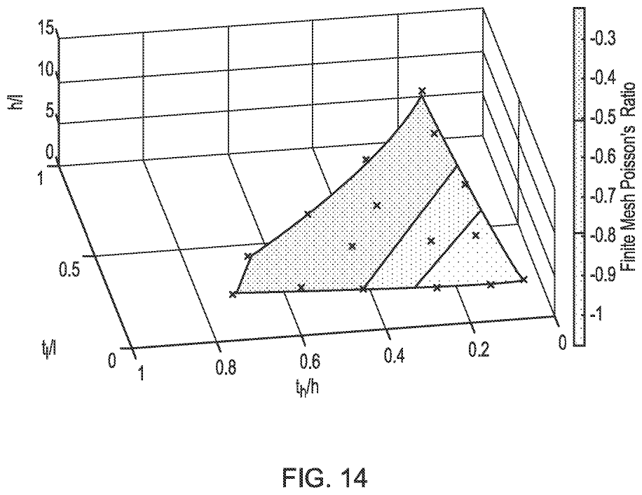

Boundary conditions imposed on the auxetic meshes by clamping both ends were shown to have strong effect in the apparent Poisson's ratios of the meshes. FIG. 14 demonstrated a 3D parameter space of h/l, tₕ/h, and tₗ/l, where a contour of a −1 Poisson's ratio was plotted for the auxetic meshes according to eq. 5. A colour map showing the measured Poisson's ratios of the simulated clamped meshes was projected onto the contour. The Poisson's ratio of the simulated meshes were observed to match the theoretical value of −1 only in the low h/l, tₕ/h, and tₗ/l corner of the contour. Away from this corner, the measured Poisson's ratios rapidly becomes less negative than eq. 5 would predict. Therefore, to ensure maximum biaxial straining performance of the meshes, thinner and longer struts are preferred.

### Device

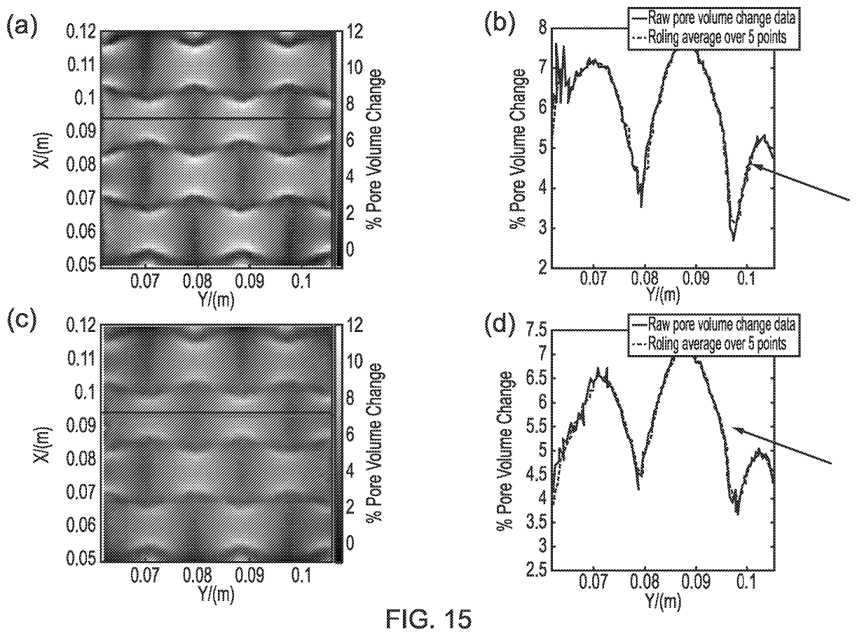

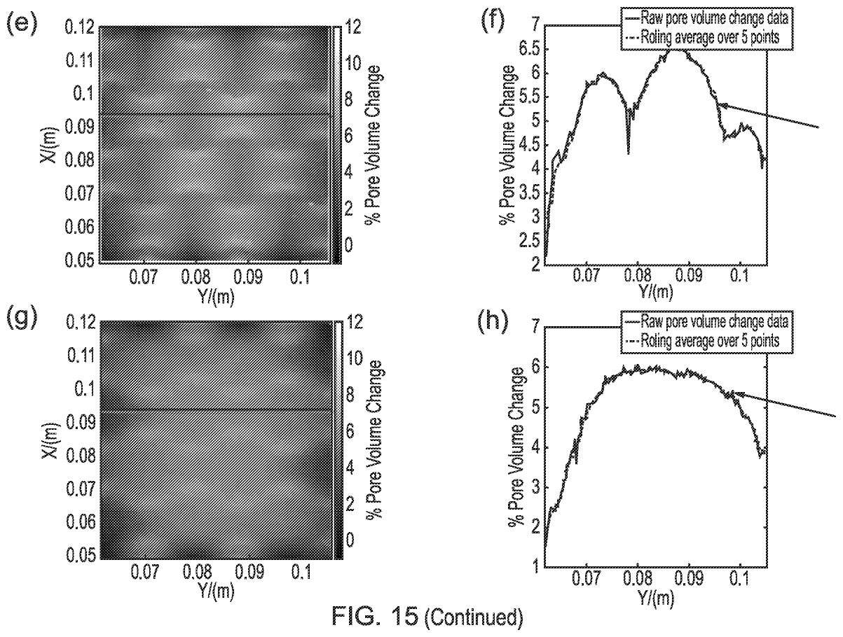

Maps of pore deformation in the scaffold at different z heights as well was corresponding line profiles for a 3% imposed strain are presented in FIG. 15. Close to the scaffold, the deformation in the pores was spatially non-uniform, due to the bonding of the scaffold to the auxetic. The percentage pore volume change in the lowest layer ranged from a high of 12% (in pores adjacent to the top and bottom of each auxetic unit cell) to a low of 3% (directly above the mesh struts). As the z height increased away from the scaffold, the deformation in the pores became gradually more uniform, until in the highest layer, the deformation in the bulk of the scaffold was 6+0.5% everywhere. This change in uniformity was reflected in the line profiles; (b) displayed troughs in percentage pore volume change corresponding to the bonding of the scaffold to the mesh, whilst (h) displayed a flat deformation plateau across the same region. Near the edges of the scaffold, clear drops in percentage pore volume change were observed. This was due to the effect of the free edges.

### REFERENCES

[1] D. M. Roper, K. A. Kwon, S. M. Best, and R. E. Cameron, “The 3D printing of freestanding PLLA thin layers and improving first layer consistency through the introduction of sacrificial PVA,” Applied Sciences (Switzerland), vol. 11, p. 6320, July 2021.
[2] J. C. Ashworth, M. Mehr, P. G. Buxton, S. M. Best, and R. E. Cameron, “Cell Invasion in Collagen Scaffold Architectures Characterized by Percolation Theory,” Advanced Healthcare Materials, vol. 4, no. 9, pp. 1317-1321, 2015.
[3] M. Nair, J. H. Shepherd, S. M. Best, and R. E. Cameron, “MicroCT analysis of connectivity in porous structures: Optimizing data acquisition and analytical methods in the context of tissue engineering,” Journal of the Royal Society Interface, vol. 17, April 2020.
[4] A. Alderson, “Modelling the effects of density variations on the in-plane Poisson's ratios and Young's Moduli of periodic conventional and re-entrant honeycombs-Part 1: Rib thickness variation project Auxetic materials for sport View project,”
[5] R. J. Mason, “Epithelial Cells: Type II Cells,” in Encyclopedia of Respiratory Medi-cine, Four-Volume Set, pp. 138-142, Elsevier Inc., January 2006.
[6] P. Andrikakou, K. Vickraman, and H. Arora, “On the behaviour of lung tissue under tension and compression,” Scientific Reports, vol. 6, 2016.
[7] R. R. Mercer, M. L. Russell, and J. D. Crapo, “Alveolar septal structure in different species,” Journal of Applied Physiology, vol. 77, no. 3, pp. 1060-1066, 1994.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The invention extends to any novel one, or any novel combination, of the features disclosed in this specification (including any accompanying claims and drawings). The claims should not be construed to cover merely the foregoing embodiments, but also any embodiments falling within the scope of the claims. Each feature disclosed in this specification (including any accompanying claims and drawings) may be replaced by alternative features serving the same, equivalent or similar purpose, unless expressly stated otherwise. Thus, unless expressly stated otherwise, each feature disclosed is one example only of a generic series of equivalent or similar features.

All of the features disclosed in this specification (including any accompanying claims and drawings) may be combined in any combination, except combinations where at least some of such features are mutually exclusive. The invention is defined by the appended claims.

1. A cell culturing apparatus comprising:
an auxetic mesh; and
a substrate for culturing cells, the substrate being attached to the auxetic mesh such that a change in at least biaxial strain in the substrate and/or the auxetic mesh occurs by the application of a uniaxial force to the auxetic mesh.

2. The apparatus according to any preceding claim, wherein the substrate comprises a three-dimensional cell culture substrate.

3. The apparatus according to any preceding claim, wherein the auxetic mesh comprises a re-entrant honeycomb structure.

4. The apparatus according to any preceding claim, wherein the auxetic mesh comprises a Poisson's ratio of −0.7 to −1.0.

5. The apparatus according to any preceding claim, wherein the auxetic mesh is embedded in the substrate.

6. The apparatus according to any preceding claim, wherein the auxetic mesh extends beyond at least two opposing surfaces of the substrate.

7. The apparatus according to any preceding claim, wherein the auxetic mesh comprises poly-lactic acid.

8. The apparatus according to any preceding claim, wherein the substrate comprises a porous structure.

9. The apparatus according to any preceding claim, wherein the substrate comprises a protein and/or a polysaccharide, optionally wherein the protein is an extracellular matrix protein.

10. The apparatus according to claim 9, wherein extracellular matrix protein is selected from the group consisting of collagen elastin, laminin, and fibronectin.

11. The apparatus according to any preceding claim, wherein the substrate is crosslinked.

12. The apparatus according to any preceding claim, wherein the substrate comprises MatriGel™.

13. The apparatus according to any preceding claim, wherein the substrate comprises cells seeded therein and/or thereon.

14. The apparatus according to claim 21, wherein the cells form a tissue and/or organoid.

15. A method for culturing cells comprising:
providing the cell culturing apparatus according to any one of claims 1 to 14; and
applying a force to the auxetic mesh.

16. The method of claim 15, wherein the force applied to the auxetic mesh comprises a physiologically-mimetic force.

17. The method according to any one of claim 15 or 16, wherein the force comprises a uniaxial force.

18. The method of according to any one of claims 15 to 17 comprising:
applying cells to the substrate;
disposing the substrate in a culture medium; and
maintaining the substrate under conditions suitable for culturing the cells.

19. A method of manufacturing a cell culturing apparatus, the method comprising:
providing an auxetic mesh; and
attaching a substrate for culturing cells to the auxetic mesh such that a change in at least biaxial strain in the substrate and/or the auxetic mesh occurs by the application of a uniaxial force to the auxetic mesh.

20. The method of claim 19, wherein providing an auxetic mesh comprises 3D-printing the auxetic mesh.

21. The method of claim 19 or 20, wherein attaching the substrate to the auxetic mesh comprises suspending a portion of the auxetic mesh with a mould and forming the substrate around the suspended portion of the auxetic mesh.
